(19) United States
(12) Patent Application Publication
(10) Pub. No.: US 2005/0106587 A1
(43) Pub. Date: May 19, 2005

(54) Method for determining of nucleic acid analytes

(75) Inventors: Holger Klapproth, Freiburg (DE); Mirko Lehmann, Freiburg (DE)

Correspondence Address: GRIFFIN & SZIPL, PC, SUITE PH-1, 2300 NINTH STREET, SOUTH, ARLINGTON, VA, 22204, US

(73) Assignee: Micronas GmbH, Freiburg (DE)

(21) Appl. No.: 10/870,986

(22) Filed: Jun. 21, 2004

Related U.S. Application Data
(63) Continuation of application No. PCT/EP02/13860, filed on Dec. 6, 2002.

(30) Foreign Application Priority Data
Dec. 21, 2001 (DE) 101 63 599.0

(51) Int. Cl. C12Q 1/68

(52) U.S. Cl. 435/6

(57) ABSTRACT

The present invention concerns generally a method as well as an apparatus for the determination of nucleic acid analytes. In particular, the present invention concerns the detection of the presence of such analytes without the conventional use of optically detectable marker substances.

The present invention generally concerns a method as well as an apparatus for the determination of nucleic acid analytes. In particular, the present invention concerns the detection of the presence of such analytes without the conventional use of optically detectable marker substances.

Essentially planar systems, called biosensors or biochips in the art, are known for use in the qualitative and/or quantitative determination of certain nucleic acid analytes, such as DNA for example. These biochips are constituted from a support, the surface of which as a rule is provided with a plurality of detection zones that are arranged for the most part in a raster configuration, wherein individual zones or groups of zones are differentiated from each other according to their specificity toward a specific detectable analyte. In the case of the determination a DNA analyte, the individual zones of the support surface contain either directly or indirectly immobilized specific nucleic acid probes, for example oligonucleotides or cDNA in mostly single-stranded form, the specificity of which toward given nucleic acids is essentially determined by the sequence order (probe design). In the context of a suitable detection method, a chip surface functionalized in this way is brought into a condition of contact with the specified DNA analytes, which in the case of the presence of target nucleic acid(s) that are detectably marked beforehand will ensure their hybridization with the immobilized probe molecules. The qualitative and optionally quantitative detection of one or more specifically constructed hybridization complexes takes place next primarily by photoluminescence measurements and mapping of the data obtained to the respective detection zones, which enables a determination of the presence of or the sequence of the nucleic acid analytes and optionally their quantification.

In addition to these luminescence-based processes, efforts have been mounted in recent years to be able to carry out DNA analysis without the need for using luminescence markers and without the required detection and imaging agents.

Thus, for example, differentiation between the possible configurations of a single strand and a double strand was attempted by means of field effect transistors (FETs) (E. Souteyrand et al., “Direct detection of the hybridization of synthetic homo-oligomer DNA sequences by field effect”, J. Phys. Chem. B., 1001, 2980, 1997) or with impedance structures (see for example P. Van Gerwen et al., “Nanoscaled interdigital electrode arrays for biochemical sensors”, Sensors and Actuators, B 49, 73-80, 1998).

A further approach from the state of the art involves the utilization of the enzymatic activity of the extracellular nuclease of Serratia marcescens, which leads to an alteration of the pH value resulting from the enzyme-mediated degradation of DNA (S. Reher, “Analysis of DNA and RNA with voltametric, potentiometric, and optical methods with the use of the extracellular nuclease of Serratia marcescens”, ISBN 3-89825-030-X, 1999).

Furthermore, there are publications that describe DNA analysis being performed with the use of certain marker substances wherein the detection is carried out without optical methods. One such approach involves marking the hybridized DNA with an electronic label and its adsorption onto a noble metal electrode, whereby this binding phenomenon can be read with an electrode. (www.microsensor.com/TechnologySystem.html, Clinical micro sensors, 2000).¹ Other work describes the coupling of a small paramagnetic body to a DNA molecule, whereby alterations in the magnetic field can be read (D. R. Baselt et al., “A biosensor based on magnetoresistance technology”, Biosensors & Bioelectronics 1998, 13(7-8):731-9, 1998).
¹This link is now dead, and points to a Motorola main page; some information is given at http://www.motorola.com/lifesciences/index.html<translator>.

Although the foregoing work highlights alternatives to luminescence-based nucleic acid analysis, they suffer from the fact that the hybridization reactions frequently take several hours to be complete, together with measurement error in the sensors used for detection, referred to in the art as “drift”. This drift leads to a signal that has been altered over time, which often cannot distinguished or clearly differentiated from the actual signal, since this latter is located within the same frequency-intensity profile as the drift. Moreover, it is generally easier to read a signal that reaches its peak height within the shortest possible time period.

The object of the present invention is the provision of an improved method through which the disadvantages connected with the drift problem will be overcome.

The problem is solved by the invention through the method according to the main claim.

According to one embodiment, the present invention comprises a method for the determination of a nucleic acid analyte through hybridization of the analyte onto a suitable nucleic acid probe immobilized on a solid phase, wherein
(a)the nucleic acid analyte is incubated with the nucleic acid probe under suitable conditions to form a hybridization complex, and
(b) the analyte is determined on the basis of physical measurement data, which is specifically associated with an enzyme-caused mass increase or decrease in the hybridization complex,
   whereby the measurement of the data is performed by at least one sensor that is an integral component of the solid phase.

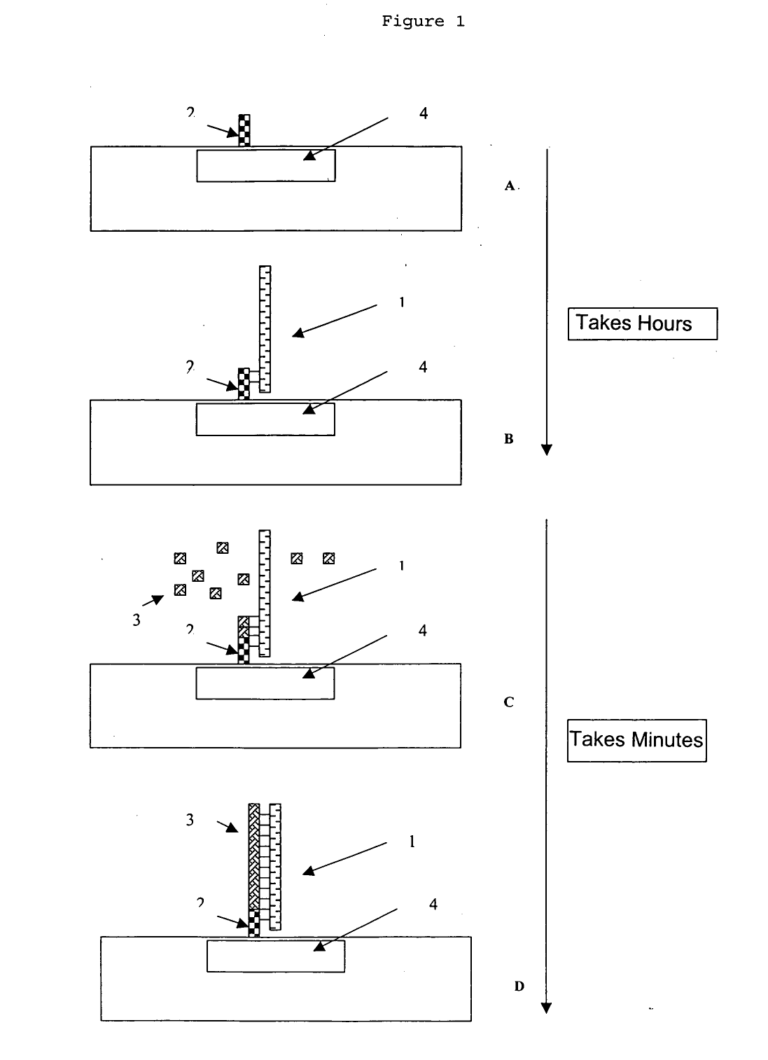

Through this indirect approach to a solution, it is possible according to the present invention to transfer the detection to another time window and thus to another frequency, which is preferably only a few seconds or a few minutes, and thus substantially to avoid the drift problem (see FIG. 1).

The preferred embodiments of this method are presented in the subclaims [sic, dependent claims].

The expression “determination” as used at present refers to any analysis of a nucleic acid and comprises in particular the detection of the presence of a nucleic acid analyte in a sample to be tested. It further comprises embodiments such as the elucidation of a nucleic acid sequence and the mapping of mutations such as particularly SNP's.² The present method thus allows for a very broad scope of possible uses since it is applicable to all determination and detection techniques available at the present time or in the future that are based upon the formation of a hybridization complex.
²SNP=single-nucleotide polymorphism<translator>.

According to one of the preferred embodiments, the enzyme that causes the mass increase or decrease in the hybridization complex can be selected from the group that includes polymerases, ligases, ribozymes, quasi-catalytic nucleic acids, DNases/RNases (exo- and endonucleases including restriction endonucleases), and RNase H, wherein a polymerase, especially a polymerase with 5′- and/or 3′-exonuclease activity, is particularly preferred.

In addition to DNA-dependent DNA polymerases, a mass increase that depends on the given constitution of the nucleic acid (RNA or DNA) can take place according to the present invention through application of RNA-dependent DNA polymerases (reverse transcriptase³) or RNA-dependent RNA polymerases (replicases). A mass increase can further be effected through the use of suitable polymerase-active ribozymes or quasi-catalytic RNAs. Either thermostable or thermolabile enzymes can be used for all of the polymerases (including ribozymes and quasi-catalytic RNAs) of the present invention.
³Source has this in the singular form, whereas all other enzymes are referred to in the plural<translator>.

An enzymatically-produced mass increase can also be effected by means of ligases. In this connection, the appropriate use of ligase-active ribozymes and quasi-catalytic RNAs of the present invention can also be indicated. Either thermostable or thermolabile enzymes can be used for all of the ligases (including ribozymes and quasi-catalytic RNAs) of the present invention.

In contrast to a mass increase, a mass decrease can also be detected. A mass decrease can take place through cleavage of the bound nucleic acids by nucleases (RNases, DNases). Either 5′- and/or 3′-exo-as well as endonucleases or RNase H can be used. Single-as well as double-strand-dependent enzymes or enzymes with both types of activity can be used. Either sequence-specific or non-sequence-specific enzymes are suitable for use as nucleases. Ribozymes and quasi-catalytic RNAs with nuclease activity are also suitable. As a rule, ribozymes and quasi-catalytic RNAs act with sequence specificity, so that the specificity can be adjusted according to need by means of a given hybridization sequence.

Accordingly, the present invention reflects the situation encountered in most solid phase-linked nucleic acid analyses, that is the presence of a single-stand nucleic acid probe immobilized on a solid phase (see FIG. 1A). Under suitable conditions, a hybridization complex that is at least partially double-stranded will be formed when a nucleic acid analyte that has a sequence that is essentially complementary to the probe sequence is present (see FIG. 1B).

According to the invention, the introduction of an enzymatic step (see FIG. 1C) takes place subsequent to the formation of the complex, whereby the action of the enzyme leads to a measurable change in the mass of this complex.

For example, should a hybridization complex be present that is composed of one of the shorter DNA probes and a comparatively longer nucleic acid analyte, a polymerase can be introduced which under suitable conditions and in the presence of the four nucleotide triphosphates (A, T, G, and C) is in a position to fill in, at least partially, the single-stranded region due to the longer nucleic acid analyte (see FIGS. 1C and 1D). Presuming a mean binding rate with a magnitude in the thousands of bases per minute, this continuous polymerization will take place within a few minutes. This example is based on the assumption that the participating hybridization partners are of different lengths, and can also be employed in the reverse case of when the analyte has a shorter chain length as compared to the probe. In this case, it can be advantageous to configure the probe design⁴ so that the probe exhibits a length of at least 100 nucleotides, and on the sensor surface approximates as closely as possible the expected singled-stranded region of the hybridization complex filled in by the action of the polymerase. This advantage can be also realized if the probe is immobilized at its 3′-end and if the filling in of the singled-stranded region takes place in the direction of the solid phase. These advantageous embodiments are not limited to polymerases but rather can be transferred to all suitable enzymes of the present invention, and depending on the desired scope of application can easily be implemented by one skilled in the art.
⁴The source word is given as ‘Sondesign’ which is either an abbreviation of or misprint for ‘Sondendesign’<translator>.

To the extent that there is no analyte present in the test sample to be analyzed that is complementary to the probe sequence, no formation of a hybridization complex will take place at this position because of the different binding energies, and because no subsequent enzyme reaction will occur, no measurement data caused by the enzyme will be recorded.

Since a hybridization usually takes on the order of several hours without the application of an electrical field, the duration of the detection of the present invention will be considerably reduced, thereby producing a much shorter time window which is more suitable for reading the sensors.

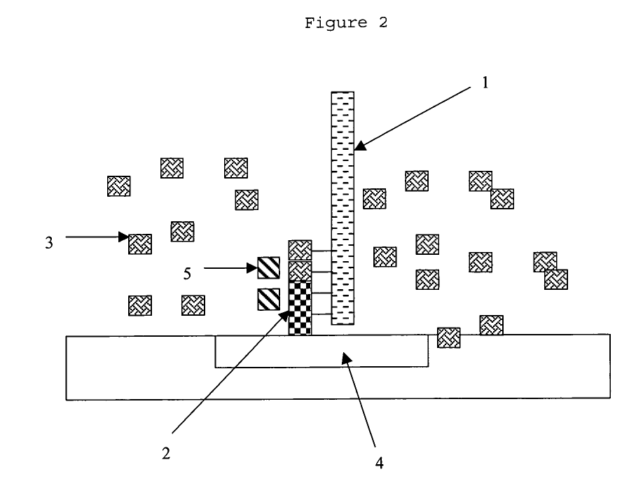

Pyrophosphate anions are produced by the action of polymerase mentioned in the above example, and are liberated during the polymerization of the nucleotide triphosphates in the single-stranded region of the hybridization complex, leading to local acidification and a corresponding drop in the pH value. These changes in the pH value can be detected through the local configuration of a pH sensor or pH detector (e.g. pH-ISFET), optimally with site specificity (see FIG. 2).

It is further envisioned according to the present invention that the pyrophosphate ions liberated by a polymerization or a ligation can also be detected indirectly, that is through a secondary enzyme cascade. For example, ATP sulfurylase and adenosine-5′-phosphosulfate (APS) participate in the first of the secondary reactions. In this case, the pyrophosphate PPi liberated during the incorporation of a nucleotide in a polymerization or ligation together with APS is converted to ATP by the ATP sulfurylase. The ATP produced thereby can catalyze further enzymatic reactions that can feed into the actual detection. For example, the ATP formed can catalyze the reaction of luciferin by luciferase, whereby a light emission is produced that can be sensed by the optical sensors of the present invention.

A modified example of the method of the present invention involves the nucleotriphosphate to be used being loaded with magnetic beads (Baselt, loc. cit., 1998) or metal particles (Clinical micro sensors, loc. cit., 2000). This loading results in the reading being amplified through the additional properties of the solid body bound to the nucleotide triphosphate. Furthermore, dyes can be located on the nucleotide triphosphate, which can be read by an integrated photodiode.

According to a preferred embodiment, at least one sensor is selected from the group including electrode structures, field effect transistors, magnetic sensors, optical sensors, and pH sensors, in accordance with the broad scope of application of the present invention.

A particularly preferred embodiment involves a combined use of different sensors of the aforementioned type. For example, the signal intensity and sharpness and thus the reliability can be optimized for a desired detection event if an apparatus suitable for the method of the present invention possesses not only one probe-specific sensor, such as for example a field effect transistor, but in addition possesses another type of probe-specific sensor, such as for example a pH-ISFET. The data obtained through this multi-parameter measurement will in optimal cases enable a more exact analysis of the signal due to the enzyme.

Furthermore, the sensor can be equipped with a heating element. Such an element could for example be constituted from circuit boards that would be activated during the CMOS⁵ process and subsequently be masked by the following layer. Temperature cycles could hereby be run, which for example could be desirable for a PCR-supported⁶ application in the scope of the method of the present invention.
⁵CMOS=“complementary metal oxide semiconductor”<translator>.

⁶PCR =“polymerase chain reaction”<translator>.

Since a batchwise addition of analytes can produce so-called “addition peaks” in the sensors, it is advantageous to run the method of the present invention continuously (flow through).

As a further aspect, an apparatus for performing the method of the present invention is provided.

This apparatus comprises a minimum of one solid phase, a minimum of one nucleic acid probe either directly or indirectly immobilized thereon, as well as a minimum of one sensor for the acquisition of physical measurement data, wherein the sensor is an integral component of the solid phase and preferably is selected from the above-defined group including electrode structures, field effect transistors, magnetic sensors, optical sensors and pH sensors.

In a preferred embodiment of the apparatus of the present invention, a plurality of different nucleic acid probes is provided by the formation of a raster-type microarray, wherein each immobilized nucleic acid probe is associated with each respective specific detection zone and especially preferably is associated with at least one sensor.

The measuring device known from EP-A-0,881,490 for the measurement of certain physiological and also morphological parameters from at least one living cell to be examined can be utilized in the present invention after appropriate modification. The described device possesses a plurality of sensors that are integral components of the support mounting on which the material to be examined is immobilized.

The support unit of the apparatus of the present invention is constituted substantially of a semiconductor material with integrated, preferably several detectors comprising a detector layer, wherein at least one of the previously described sensors is incorporated as the detector, optionally in combination (see above). Furthermore, the support unit can possess heating elements in order to be able to provide different temperatures during use (see above). In a particularly preferred embodiment, signal processing takes place at least partially within the provided sensor chip(s).

According to one aspect of the present invention, the sampled measurement data for example can be analyzed directly on the chip with analog circuits, in which for example a value is sampled each millisecond, which then could also be compared against a reference value from a measurement carried out previously, which may also be stored on the chip. Moreover, it would be possible in this manner to subtract nonspecific interference signals such as for example interspersed external signals.

To the extent that the sensor surface possesses the design of a microarray configuration in which a plurality of detection fields are evaluated, the detection of the measuring field or measuring point signal value can take place sequentially, in which for example entire lines or columns of the sensor surface or portions of the same can be detected one after the other (multiplex application).

By way of example, the electronic output signal of the detector can be conveyed to an external analysis device after an analog-digital conversion by means of appropriate circuitry systems (see above).

In order to be able to carry out the method of the present invention with this layer of sensors, in a further preferred embodiment it can coated with a substance capable of coupling. Typically here the sensor chip surfaces, such as from silicon dioxide for example, are immersed in a solution of bifunctional molecules (so-called “linkers”), that include for example halosilyl (e.g. chlorosilyl) or alkoxysilyl groups for coupling to the support surface, so that a self-organizing monolayer (SAM) forms, through which covalent bonds will be produced between the sensor surface and the receptor. By way of example, the coating can be performed with glycidyltriethoxysilane, for example by dipping [the support] in a solution of 1% of the silane in toluene, slowly withdrawing it, and carrying out immobilization by “baking” at 120° C. A coating prepared in this manner will generally exhibit a thickness of a few Ångströms. The coupling between the linker and the receptor molecule(s) takes place at a suitable further functional group, for example an amino or epoxy group. Suitable bifunctional linkers for coupling a plurality of different receptor molecules, especially those of biological origin, to a plurality of support surfaces are well known in the art, and examples can be found by reference to “Bioconjugate Techniques” by G. T. Hermanson, Academic Press 1996. Regarding the formation of thin polymer layers as a coupling matrix for the creating of a functionalized surface, reference can be made to WO 00/43539. The nucleic acids envisioned probe molecules in the present invention can subsequently be applied by means of a conventional pressure apparatus and be immobilized.

By using established methods, hybridizations with for example DNA can be carried out on surfaces prepared in this manner. This can be carried out for example by means of PCR. During the hybridization, the DNA analyte will bind to the complementary strand of the probe present on the sensor (provided it is present). Positive hybridization events can be detected through the use of the method of the present invention.

The measurement of site specific mass increases can be carried out by physical methods. For example, measurements can be made of site-specific changes in the refractive index, site-specific changes in the electrical resistance or electrical conductivity, site-specific changes in optical density, or site-specific changes in the dichroic effect, etc.

Fundamentally, the general method of the present invention is suitable for a broad spectrum of areas of application, wherein a differentiation can be made between the purely diagnostic detection of specific analytes in a sample to be tested on the one hand, and the complex modifications of the method for the elucidation of sequence data or information on functional correlations in the context of research problems in genomics. This differentiation is merely by way of illustration and does not in any way limit the fundamentally broad utility of the method of the present invention.

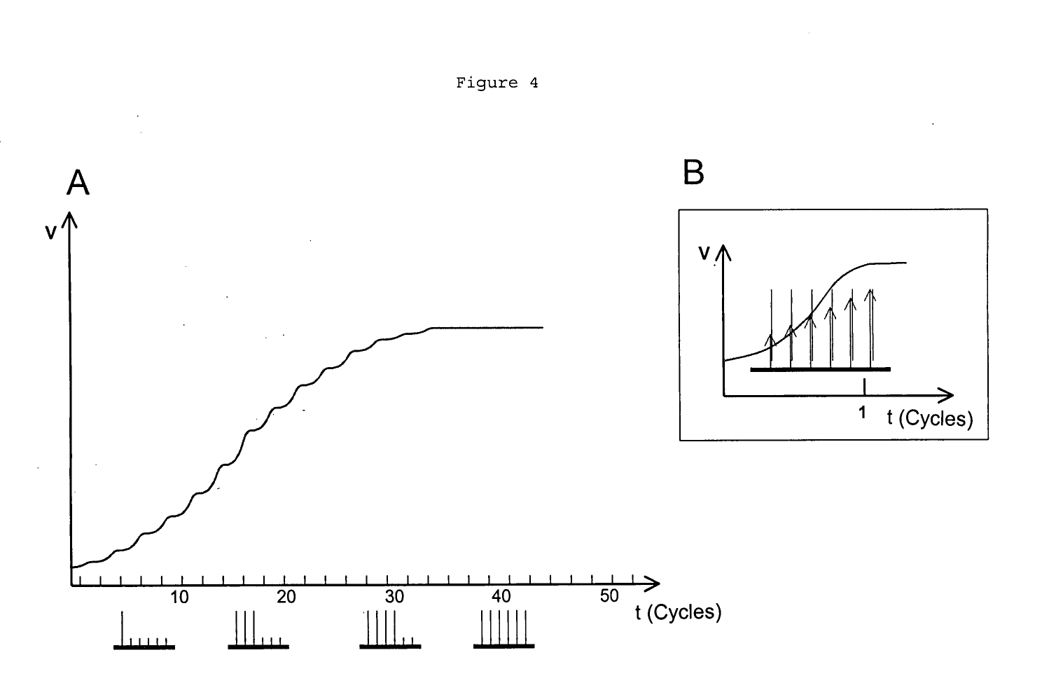

For example, the method of the present invention is especially suitable for the determination of DNA sequences, which can be carried out by means of parallel amplification through nested PCR, preferably in a combined liquid phase/solid phase DNA microarray system, since in this manner both the use of modified nucleotides such as for example [modified with] biotin or digoxiginin, as well as the conventionally used fluorescent dyes and other marker substances can be avoided. Nested PCR in a combined liquid phase/solid phase DNA microarray system (see FIG. 4) possesses the same sensitivity as a conventional PCR, that is PCR carried out in the liquid phase, but it also provides higher specificity than the conventional hybridization assays and primer extension assays. This advantage results from the fact that its own specificity for the primer/test DNA/polymerase with respect to the amplification, and additionally through the specific correlation between the internal PCR primer immobilized on the solid support (which thus also functions as a probe) and the amplicon, is significantly increased. Overall this results in a specificity that surpasses that of for example a 5′-exonuclease assay (for example using TaqMan™-Polymerase).

The sensor signals are captured with a recording unit. The recording unit possesses a very fast converter for conversion of the analog detector signals into digital values, which are stored in memory. An analysis of the digital values is preferably carried out in real time, but can also be performed with a time delay. An ordinary microprocessor can be used for the analysis of the digital values.

The invention and advantageous embodiments will be further clarified with the help of the Figures:

FIG. 1 shows schematically the sequence of events of an embodiment of the method of the present invention. (A) The nucleic acid probe (2) is covalently bound to the surface. (B) After addition of the nucleic acid analyte (1), a hybridization complex is formed, usually within the time frame of several hours. (C) By using a suitable enzyme, such as for example a polymerase, in the presence of the four nucleotides A, T, G and C (in the case of DNA), the single-stranded region of the complex is filled in within a very short period of time of a few minutes (D), whereby a signal is generated much more rapidly and is read by a sensor that is an integrated component of the solid phase.

FIG. 2 illustrates the principle of a preferred embodiment through the use of a polymerase reaction which liberates phosphate ions (5), leading to a local change in the pH value. This change can be recorded by an integrated sensor (4).

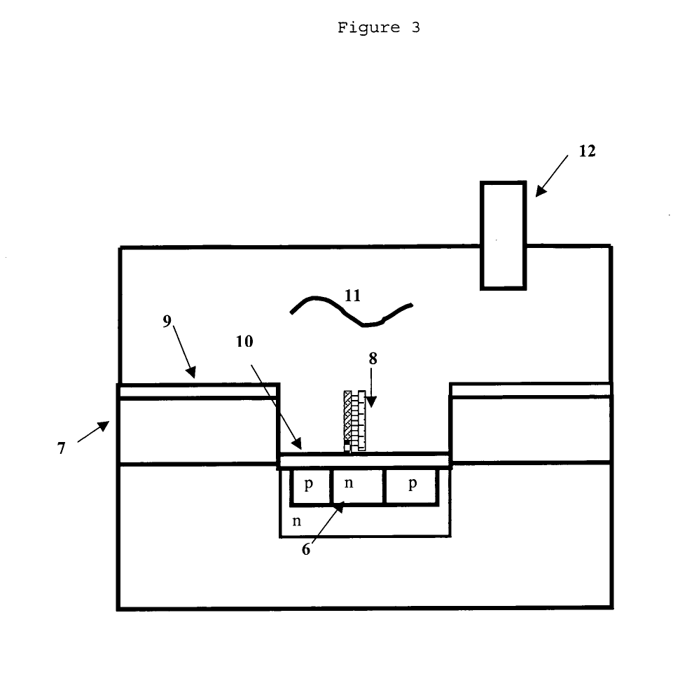

FIG. 3 shows a field effect transistor fabricated in the course of a CMOS process. The field effect transistor comprises a p-n-p layer in an n-well with a thin isolator (10) (for example 10 nm thermal oxide) located on the surface, to which the nucleic acid probe is applied either directly or indirectly, which then undergoes hybridization. A preferred embodiment has the protective layer (7) in the vicinity of the field effect transistor etched down either with a sharp edge or in a stepped fashion, so that the process of the hybridization and mass increase (8) take place in a recessed zone. The surface of the apparatus can affect the hybridization of the nucleic acid molecules either actively or passively through application of, for example, noble metal hydrophilic/hydrophobic materials (9). In a measurement solution (11), such as for example 1 M NaHCO₃, one can measure the change in the dielectric properties of the gate which occur as a result of the filling in of the single-stranded region of the hybridization complex. The accompanying shift in the flat band potential can be read with a field effect transistor by using a reference electrode (12) located in the solution. Examples of the signal to be recorded include the current between the drain and source electrodes, or the voltage between the reference and source electrodes (see for example B. Palan et al., “Fundamental Noise Limits of ISFET-Based Microsystems”, Poster-Beitrag 4P26, EUROSENSORS XIII (ISBN 90-76699-02-X), S. 169 ff., 1999).

FIG. 4 shows the change in voltage sampled with an FET in the course of a parallel amplification based on the so-called “Nested on Chip” PCR (NOC PCR, see above). Part A of the Figure shows the voltage change at a probe position over the course of the entire NOC PCR. The X-axis gives the number of cycles, while the measured voltage is given on the Y-axis. Beneath the X-axis, the primer molecules (=probe molecules) coupled to the probe position are symbolized: only a few primers are elongated in the first cycle, followed by a strong exponential increase in the middle cycle and an increasing saturation (essentially all primers having been elongated) in the later cycles. The curve shows that the measured voltage becomes higher with increasing mass at the probe position. Part B of the Figure shows the course of the voltage within a single cycle (median cycle number). In addition to the primer, the template and the elongation of the primer from left to right are also shown here. It is apparent from this illustration that the voltage becomes higher in proportion to the elongation of the primer

The invention will be further clarified in the following with examples.

### Fabrication of the Sensor Chips of the Present Invention

The CMOS sensor is produced on 5″ or 6″ wafers with a 1.2 μm CMOS process. Each field effect transistor is located on a p-substrate in an n-well. The implantation of the drain and source regions takes place after the field oxidation. A thermal gate oxide with a thickness of ca. 10 nm is applied. The gate is protected with polycrystalline silicon during the following process steps. Next, a silicon dioxide layer is applied and structured with the use of a chemical vapor deposition (CVD) process. Aluminum is sputtered on and likewise structured. Passivation is achieved with an Si₃N₄ PECVD⁷ nitride layer and a CVD SiO₂ layer. The gate isolator is unmasked in the next etching step.
⁷PECVD=plasma-enhanced chemical vapor deposition<translator>.

### Coating the CMOS Sensor

The CMOS sensor fabricated as above is dipped in a solution of 1% GOPS (glycidoxypropyltriethoxysilane) and 0.1% triethylamine in toluene for a period of ca. 2 hours to coat it with the silane. The chip is next removed from the solution and is fixed by prompt drying at 120° C. in a drying oven for a period of approximately 2 hours.

The chip thus coated is optimally stored under exclusion of moisture until the bioconjugation.

### Bioconjugation with Oligonucleotide Probes

Contactless printing by conventional techniques is used to apply 5′-amino-modified oligonucleotide probes to the above coated chip. For this purpose, a 5 μM solution of the oligonucleotide probes in PBS⁸ buffer is prepared. After the printing, the coupling reaction proceeds at 50° C. in a humid chamber. The chips are subsequently rinsed with distilled water and then washed with methanol to dryness. Any remaining solvent residue is subsequently removed through evaporation in the fume hood.
⁸PBS=phosphate-buffered saline<translator>.

### Specimen collection

Fragments of the hemochromatosis gene from human DNA isolates were amplified with PCR. Suitable primer sequences were used in the amplification, for example such as described in the patent U.S. Pat. No. 5,712,098.

The reaction mixture included the following standard reagents—primer: 0.5 μM; dATP, dCTP, dGTP: 0.1 mM; dTTP: 0.08 mM; PCR Buffer; MgCl₂: 4 mM; HotStarTaq (Perkin Elmer) 2 units (50 μL). During the PCR reaction (35 cycles, 5 min 95° C., 30 sec 95° C., 30 sec 60° C., 30 sec 72° C., 7 min 72° C.), the available nucleotides were incorporated into the newly synthesized DNA. Subsequently, single-stranded DNA was generated by addition of T7 Gene 6 exonuclease (100 units/50 μL PCR preparation) and heating of the preparation (30 min 37° C., 10 min 85° C.).

### Hybridization

The above reaction mixture was hybridized on the chip in a buffer 5×SSPE,⁹ 0.1% SDS¹⁰ (12 μL) under a cover glass for a period of 2 hours at 50° C. in a humid chamber.
⁹SSPE=saline sodium phosphate EDTA (ethylenediaminetetraacetic acid)<translator>.

¹⁰SDS=sodium dodecyl sulfate<translator>.

Subsequently, the chip was rinsed with 2×SSPE 0.1% SDS and cleaned by washing with water.

1. A method for the determination of a nucleic acid analyte by hybridizing the analyte on a suitable nucleic acid probe immobilized on a solid phase, wherein
(a) the nucleic acid analyte is incubated with the nucleic acid probe under suitable hybridization conditions to form a hybridization complex, and
(b) the analyte is determined on the basis of chemical/physical measurement data, which is specifically associated with a mass increase or decrease in the hybridization complex that is due to an individual enzymatic step,
whereby the measurement of the data is performed within a few seconds or minutes by at least one sensor, the sensor being an integral component of the solid phase and being selected from the group including electrode structures, field effect transistors, magnetic sensors, optical sensors, pH sensors, and combinations of the same.

2. The method recited in claim 1 wherein the steps (a) and (b) are carried out continuously (flow through).

3. The method as recited in claim 1, wherein the enzyme that effects the mass increase or decrease in the hybridization complex is selected from the group that includes polymerases, ligases, ribozymes, quasi-catalytic nucleic acids, DNases, RNases, and RNase H.

4. The method as recited in claim 1, wherein polymerase is selected as the enzyme.

5. The method as recited in claim 4, wherein the polymerase exhibits 5′- and/or 3′-exonuclease activity.

6. An apparatus for performing the method as recited claim 1, comprising a minimum of one solid phase, a minimum of one nucleic acid probe either directly or indirectly immobilized thereon, as well as a minimum of one sensor for recording the chemical/physical measurement data, wherein the sensor is an integral component of the solid phase.

7. The apparatus as recited in claim 6, wherein the minimum of one sensor is selected from the group including electrode structures, field effect transistors, magnetic sensors, optical sensors, and pH sensors, and combinations thereof.

8. The apparatus as recited in claim 6, wherein a plurality of different nucleic acid probes are arranged in a raster configuration forming a microarray.

9. The apparatus recited in claim 6, wherein each immobilized nucleic acid probe is associated with a minimum of one sensor.

10. The apparatus as recited in claim 6, comprising in addition a minimum of one heating element that is associated with a minimum of one sensor.
